(19) United States
(12) Patent Application Publication
(10) Pub. No.: US 2015/0216483 A1
(43) Pub. Date: Aug. 6, 2015

(54) BLOOD PRESSURE ESTIMATION BASED ON NEURAL ACTIVITY

(71) Applicant: NERVEX A/S, Aalborg Øst (DK)

(72) Inventors: Cristian Sevcencu, Aalborg O (DK); Johannes Jan Struijk, Terndrup (DK); Thomas Nørgaard Nielsen, Aalborg (DK); Kristian Rauhe Harreby, Gistrup (DK)

(21) Appl. No.: 14/429,524

(22) PCT Filed: Sep. 19, 2013
(86) PCT No.: PCT/DK2013/050299

§ 371 Date: Mar. 19, 2015

(30) Foreign Application Priority Data
Sep. 19, 2012 (DK) PA 2012 70576

(51) Int. Cl. A61B 5/00; A61N 1/36; A61B 5/04; A61M 5/172; A61B 5/021; A61B 5/0402

(52) U.S. Cl. CPC A61B 5/7278; A61B 5/021; A61B 5/0402; A61B 5/04001; A61M 5/1723; A61N 1/36117; A61N 1/36135; A61M 2205/3303; A61M 2230/30

(57) ABSTRACT

A device and a method for analyzing an input signal (1) representing a neural activity of a nerve containing nerve fibers originating from baroreceptors from a patient. E.g. neural activity recorded with an electrode implanted to detect Vagus nerve activity. The invention is based on the insight that this neural activity can be used to determine a value indicative of the patient's blood pressure (BP). An ECG signal (2) can be used for deriving a trigger feature for processing of the nerve activity data, e.g. to arrive at a profile of the patient's Blood Pressure Pulse. E.g. the device can include a Vagus nerve stimulation unit which is controlled in response to an output from the calculation of blood pressure (BP), and/or features related to the Blood Pressure Pulse.

### FIELD OF THE INVENTION

The present invention relates to a medical device for estimating blood pressure (BP). Moreover, the invention relates to a computer program product and to a method for detecting the blood pressure pulses and extracting BP related information from neural activity.

### BACKGROUND OF THE INVENTION

Approximately 1 billion patients worldwide are affected by hypertension, which represents a major risk factor for heart and renal failure, stroke, and myocardial infarction. About 10% of these patients do not respond to anti-hypertensive drugs and suffer from resistant hypertension (RH).

It is known in the art that stimulation of the vagus nerve or the carotid sinus can reduce BP. Information about the BP can therefore be used in a closed-loop system to ensure efficient and safe stimulation for the treatment of RH.

Alternative treatments for hypertension and heart failure, including electrical stimulation of autonomous nerves, and methods for assessing the BP in order to control such treatments have received considerable interest over the preceding decades. Nevertheless, there is still no method available for assessing BP with implantable devices.

### SUMMARY OF THE INVENTION

The invention is directed towards methods and devices for estimating components of central BP, such as the pulse pressure and the systolic or diastolic pressure. This information can be used for the control of anti-hypertensive treatments, such as Vagus Nerve Stimulation (VNS), carotid sinus stimulation or timed delivery of anti-hypertensive drugs, as well as for other treatments that affect BP, such as VNS for treatment of patients in risk of heart failure.

In a first aspect, the invention provides a device arranged to analyze an input signal representing a neural activity of a nerve containing nerve fibers originating from baroreceptors from a patient, such as an input signal representing neural activity of the Vagus nerve of a patient, and to determine a value indicative of the patient's blood pressure BP based thereon, such as a value indicative of the patient's systolic blood pressure.

Especially, it may be preferred that the input signal represents a neural activity of the patient's Vagus nerve. At least a part of the device, e.g. the entire device, may be arranged for implantation into the patient. Preferably, the device comprises a processor arranged to execute a control algorithm, wherein the control algorithm comprises preprogrammed steps serving to analyze the input signal.

The inventors of the present invention have realized that a neural signal from a nerve that transmit information from baroreceptor fibers can be used to produce a signal that varies with the arterial pressure pulses, thus providing information on peaks during the contraction of the heart which is related to the arterial BP. From this signal the arterial pressure pulses can be detected and BP parameters can be extracted, which is indicative of pulsatile and tonic BP.

Various aspects of the invention are defined in the appended claims. It is to be understood that the various aspects, device, method, and computer program code, as well as method of treatment may be combined in any way.

The invention is advantageous, since it is possible to reliably determine a patient's blood pressure BP by use of the neural activity of e.g. the vagus nerve. Thus, for patients with an implanted vagus nerve electrode used for Vagus Nerve Stimulation (VNS), it is possible to use the same electrode also for obtaining a measure of the patient's blood pressure, and thus provide an input to a VNS system, e.g. in a closed-loop configuration, i.e. an “on-demand” type of configuration for controlling the VNS in response to a measured BP to provide the necessary VNS to obtain a target BP.

The general level of activity in the neural signals ElectroNeuroGram (ENG) recorded from nerves that contain afferent nerve fibers from baroreceptors may display variations that can be correlated to changes in the BP. However, neural signals may comprise signals related to a number of functions that the nerve is involved in, e.g. relating to various organs. Although, a BP increase may result in increased activity of, e.g., the vagus nerve, an increase in general nerve activity is for this reason not specific to BP increases. The present invention utilizes the pulsatile nature of the BP in the central arterial vessels where the baroreceptors are located to emphasize components of the nerve signal that vary with these BP Pulses (BPP) and filter out neural activity that does not vary with the BPP. This enables extraction of BP specific information from a mixed neural signal and may enable patients to be continually monitored for BP alterations.

In some embodiments, the device may be implemented as a unit for determining BP. In other embodiments, the device may form part of a closed loop BP control system, e.g. where a combined sensing and stimulation electrode is attached to the vagus nerve. Such an electrode can either be implanted in the nerve (e.g. an intra-neural or an intra-fascicular electrode), or mounted on the nerve (e.g. a cuff electrode). In either situation, the electrode may comprise a sensing unit and a separate stimulation unit. Alternatively, sensing and stimulation may be performed using one and the same electrode unit which serves in a sequential order for sensing and stimulation. In such closed loop BP control system, a sensed BP is input to an algorithm which generates an output accordingly to a stimulator unit, which then generates an electric stimulation signal to the vagus nerve electrode to influence the patient's BP.

The BP estimate provided by the device may be used alone or in combination with other features for the control or adjustment of a treatment modality. In some embodiments, the device may e.g. combine BP and heart rate (HR) limits to ensure safe stimulation.

Whether the device is implemented as a “stand-alone” implant for determining BP, or is implanted as part of an “on-demand” (closed-loop) BP control system, calibration of the device relative to the patient's BP is done intra-operatively by manipulating the patient's BP within a range of BP values and calibrating the device accordingly. Calibration of the device is performed relative to the patient's BP values which can be measured using conventional means, such as an arm-cuff or a catheter based tensiometer. As the implanted device may be equipped with a wireless communication system, re-calibration of the device and adjustment of the stimulation paradigms can periodically be performed using external equipment utilizing such wireless communication system.

In addition to the embodiments described above, the device may be used during clinical sessions for evaluation and adjustment of devices that e.g. stimulate baroreceptor fibers. Treatment induced decreases in BP may coincide with alterations in HR, which can then be used to confirm that the target nervous tissue is being activated. However, when the BP decrease does not coincide with HR alterations evaluation of stimulation settings may be difficult since e.g. arm cuff measurement of BP is too slow to record transient BP changes. By recording signals that originate from baroreceptors, the immediate effect of stimulation on central arterial BP may be evaluated.

It is to be understood that the device and method can be applied to a “patient” meaning a person or an animal.

In the following, some embodiments of the device are defined.

The device may comprise an input unit arranged to receive the input signal and to output a digital version thereof.

The device may comprise a signal processing unit arranged to analyze the digital version of the input signal and to extract a segment of the neural activity data represented therein, to emphasize variations occurring with the cardiac rhythm. Especially, the signal processing unit may be arranged to generate an averaged segment based on a plurality of segments of the neural activity data.

The device may comprise an estimation unit arranged to estimate one or more blood pressure components from Blood Pressure Pulse information in the neural activity data represented in the input signal.

The device according may be arranged to extract an envelope (VENG) of a neural activity data represented in the input signal. Especially, said envelope may be extracted by determining at least one of: a positive peak, a negative peak, and an amplitude, of the neural activity data represented in the input signal. The device may be arranged to calculate Blood Pressure Pulse information in response to said envelope of the neural activity signal Blood Pressure

The device may comprise means for performing a calibration procedure, so as to increase precision of the value indicative of the patient's blood pressure.

In an embodiment, the device comprises:
a signal processing unit arranged for preprocessing the neural signal to emphasize Blood Pressure Pulse information,
a signal extraction unit arranged extracting one or more Blood Pressure Pulse-related feature(s) from either a neural signal segment or an averaged neural signal segment, and
an estimation unit arranged for calculating from the at least one of the extracted one or more Blood Pressure Pulse-related feature(s), an estimated value of one or more Blood Pressure components.

The signal processing unit may be arranged for detecting a trigger feature marking each Blood Pressure Pulse events. Especially, the signal processing unit is arranged to detect the trigger feature from an ElectroCardioGraphy input signal, or from the input signal. The signal extraction unit may be arranged to perform a segmentation of the input signal, and to generate an averaged neural signal segment in response to an output of said segmentation.

In a specific embodiment, the device comprises:
an input unit for receiving and digitizing a neural signal, and an Electro CardioGraphy signal,
a signal processing unit for:
   preprocessing the neural signal to emphasize Blood Pressure Pulse information,
   detecting from the processed neural signal a trigger feature marking each Blood Pressure Pulse,
   detecting at least one feature from the Electro CardioGraphy signal, and
   in response to detecting a trigger feature, extracting a segment of processed neural signal around the trigger feature, and
a signal extraction unit for:
   generating an averaged signal segment from a number of nerve signal segments, and for
   extracting one or more Blood Pressure Pulse-related feature(s) from either the neural signal segment or the averaged neural signal segment, and
an estimation unit for:
   calculating from the extracted Blood Pressure Pulse-related feature(s) an estimated value of one or more Blood Pressure components,
   calibrating the system by extracting Blood Pressure-related feature(s) and estimating the value of one or more Blood Pressure components in normal Blood Pressure conditions to establish preset values for Blood Pressure components in normal conditions, and for
comparing the estimated value of the Blood Pressure component(s) to preset intervals for use in a treatment modality.

Parts of or all of a preprocessing of the input signal may be performed by means of hardware components.

Information from an ECG may be used to assist in identification of Blood Pressure Pulse.

Blood Pressure Pulse onset may be used to trigger onset of a treatment modality, and/or an ElectroCardioGraphy signal is used to trigger onset of a treatment modality.

The nerve activity data may be obtained from at least one nerve selected from: The vagus nerve (cranial nerve X), the glossopharyngeal nerve (cranial nerve IX), The carotid sinus nerve (Hering's nerve), and The aortic depressor nerve.

Nerve activity data may be recorded from the whole nerve, or from part of the nerve.

The device may further comprise or be connected to a treatment modality or equipment arranged for treatment of the patient. The treatment modality may comprise a Vagus Nerve Stimulation system or a drug-delivery system.

The device may comprise a nerve electrode arranged to sense said neural activity, e.g. a cuff electrode arranged for being mounted around a patient's Vagus nerve.

The device may comprise a neural activity recorder arranged to record a sequence of a neural signal.

The device may comprising a nerve stimulation unit arranged to generate an electric nerve stimulation signal. Thus, such device may be capable of provide a electric nerve stimulation, e.g. to the Vagus nerve, so as to control a patient's blood pressure. In some embodiments, a control unit is included which is arranged to control the nerve stimulation unit in response to the determined value indicative of the patient's blood pressure with the purpose of controlling the patient's blood pressure. Thus, in such embodiment utilizes the closed-loop or “on-demand” principle, and the inventive way of obtaining a value indicactive of the patient's blood pressure from neural activity is advantageous, since it is easy to provide implantable devices being able to function in a closed-loop manner. Especially, the control unit may be arranged to control the nerve stimulation unit in response to one or more Blood Pressure Pulse-related (BPP-related) features. More specifically, the control unit may be arranged to control the nerve stimulation unit so as to adjust a strength of the electric nerve stimulation signal relative to an amplitude of a BPP-related feature. This BPP-related feature may comprise a BPP-related profile. The BPP-related profile is calculated based on at least one of: a rectified signal, a calculated signal power, a calculated variance, a calculated entropy, or a frequency dependent feature. As mentioned, the nerve stimulation unit and the control unit may be connected to form a closed-loop configuration. The device may comprise a wireless receiver that allows the device to receive a wireless external control signal to influence a function of a processor arranged to execute a control algorithm serving to analyze the input signal, and/or serving to control a nerve stimulation unit. Thus, in case the device is arranged for implantation, it is possible to wirelessly control the function of the analyzing and/or control or adjust one or more parameters of the nerve stimulation unit without the need for performing any surgical steps on the patient.

In a second aspect, a method for estimating a blood pressure of a patient, comprising
analyzing neural activity data of a nerve containing nerve fibers originating from baroreceptors, from a patient, according to an analyzing algorithm, and
determining a value indicative of the patient's blood pressure (BP) based thereon.

In one embodiment, the method comprises determining an envelope of a nerve signal, e.g. determining an envelope of a second powered version of a nerve signal.

The method may comprise deriving a trigger feature from ElectroCardioGraphy (ECG) data from the patient temporally aligned with the neural activity data. Especially, the method may comprise processing the neural activity data by emphasizing Blood Pressure Pulse information by means of said trigger feature.

The method may comprise determining a measure of a systolic blood pressure of the patient based on said neural activity.

The method may comprise filtering the neural activity data, such as low pass filtering a sequence of a neural signal prior to determining the value indicative of the patient's blood pressure.

The method may be implemented fully in software, however it may be preferred that parts of the method may be performed in hardware, and other parts in software.

The device and method may form part of a hypertension treatment.

In a third aspect, the invention provides a computer executable program code arranged to perform the method according to the first aspect.

In a fourth aspect, the invention provides a method of treating hypertension using the device according to the first aspect, or the method according to the second aspect.

It is appreciated that the same advantages and embodiments of the first aspect apply as well for the second, third, and fourth aspects. In general, the first, second, third, and fourth aspects may be combined and coupled in any way possible within the scope of the invention. These and other aspects, features and/or advantages of the invention will be apparent from and elucidated with reference to the embodiments described in the following.

### BRIEF DESCRIPTION OF THE FIGURES

Embodiments of the invention will be described, by way of example only, with reference to the following drawings:

FIG. 1 illustrates a device for BP estimation according to an embodiment of the invention,

FIG. 2 shows a diagram of the elements of a device for BP estimation based on neural signals,

FIG. 3 shows an example of data recorded in an experimental study in anaesthetized female pigs and illustrates the correlation between the processed neural signal and the BP and respiration signals, respectively,

FIG. 4 shows an example of cardiovascular profiles obtained from the experimental study, as generated from the BPP trigger signal, of recorded arterial BP, and a neural signal processed by two alternative preprocessing algorithms,

FIG. 5 shows recorded systolic BP and BP estimated from a neural signal obtained from the experimental study,

FIG. 6 shows systolic BP, diastolic BP, and the feature for BP estimation averaged across seven pigs relative to a BP perturbation at time 0,

FIG. 7 shows examples of stimulation paradigms when the device is implemented as part of an “on-demand” VNS or carotid body stimulation system for BP control, and

FIG. 8 illustrates a diagram of steps of a method embodiment.

### DETAILED DESCRIPTION OF EMBODIMENTS

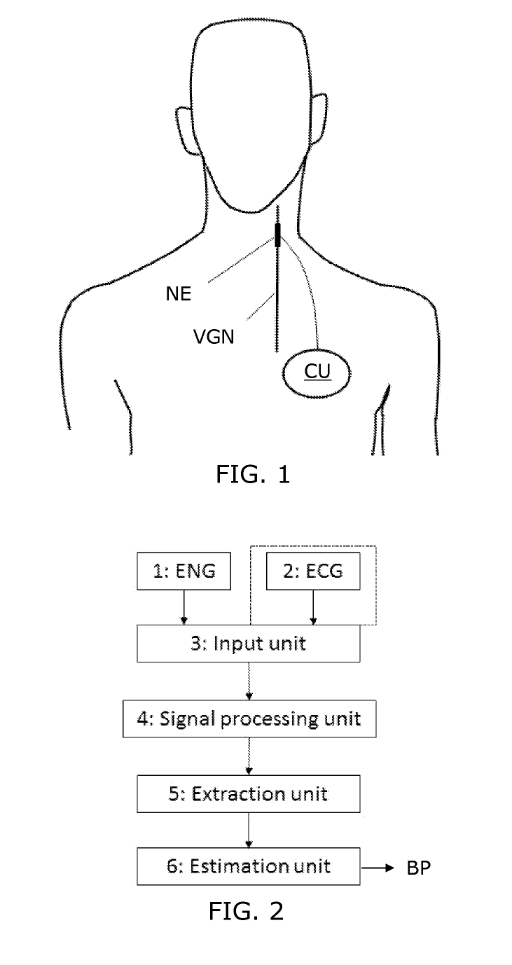

FIG. 1 shows an embodiment which may be implemented in an implantable system. The system or device comprises an electrode NE that records neural activity ElectroNeuroGram (ENG) from a nerve transmitting information from baroreceptors in central vessels, e.g. the vagus nerve VGN, and a computation unit CU that is connected to the electrode NE and receives the neural signal from the electrode NE, digitizes it, and implements the methods of the invention. A system embodiment comprising a treatment modality would require further components, e.g. a stimulator unit, e.g. contained in the same physical “box” as the computation unit CU, and a stimulation electrode, which could be the same physical electrode as the recording electrode NE, however in other versions, the stimulation electrode may be a separate electrode positioned at another location than the recording electrode NE, or positioned at the same location as the recording electrode NE. Both the electrode NE and the computation unit CU may be arranged for implantation into the patient.

FIG. 2 shows elements of a device embodiment in which a neural signal 1 is received, e.g. from the vagus nerve, or a pre-recorded nerve activity sequence, and this signal 1 is treated in such a way as to emphasize the variations that occur with the cardiac rhythm, namely by means of receiving an ECG input signal 2. An input unit 3 serves to digitize the input signals 1, 2. The digitized versions of the two input signals 1, 2 are processed by a signal processing unit 4, an extraction unit 5, and an estimation unit 6, which finally outputs a measure BP indicative of the patient's blood pressure. The device may in addition to, or alternative to the blood pressure output BP, be arranged to output one or more measures indicative of the patient's Blood Pressure Pulse, e.g. information related to the Blood Pressure Pressure profile, e.g. for use as control of a nerve stimulation unit.

In a specific version of the device in FIG. 2, the input unit 3 can be arranged to receive and digitize the neural input signal 1, and possibly an Electro CardioGraphy signal 2. The signal processing unit 4 is arranged for preprocessing the neural input signal 1 to emphasize Blood Pressure Pulse information, for detecting from the processed neural signal a trigger feature marking each Blood Pressure Pulse, for detecting at least one feature from the Electro CardioGraphy signal 2, such as R-peaks, and in response to detecting a trigger feature, extracting a segment of processed neural signal around the trigger feature. The signal extraction unit 5 can be arranged for generating an averaged signal segment from a number of nerve signal segments, and for extracting one or more Blood Pressure Pulse-related feature(s) from either the neural signal segment or the averaged neural signal segment. The estimation unit 6 is can be arranged for calculating from the extracted Blood Pressure Pulse-related feature(s) an estimated value of one or more Blood Pressure components, calibrating the system by extracting Blood Pressure-related feature(s) and estimating the value of one or more Blood Pressure components in normal Blood Pressure conditions to establish preset values for Blood Pressure components in normal conditions, and for comparing the estimated value of the Blood Pressure component(s) to preset intervals for use in a treatment modality.

It is to be understood that the neural input signal 1, and the optional ECG input 2, can be provided to the device as digital data, and thus.

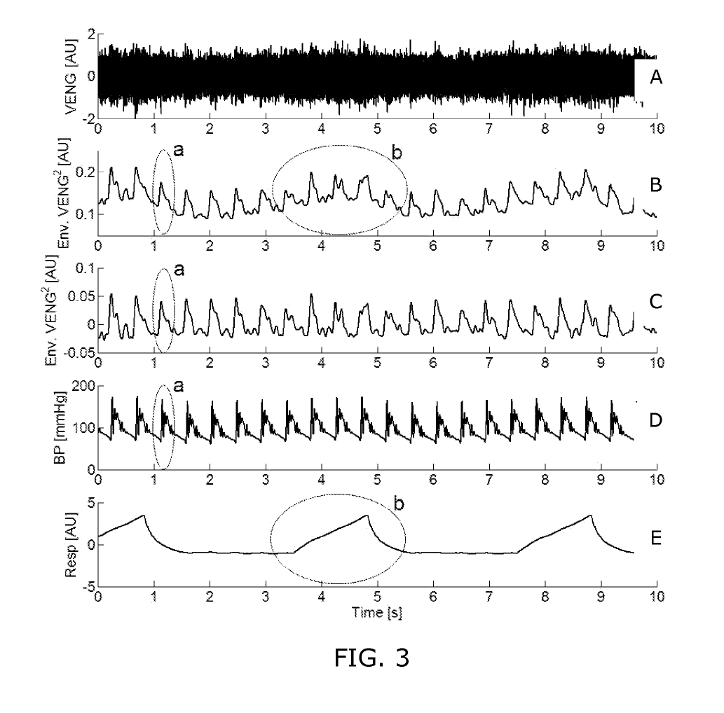

FIG. 3 shows an example of real data recorded from a pig. In FIG. 3, A is a 10 s segment of the raw vagus nerve signal (VENG). In FIG. 3, B is the same 10 s after the VENG has been processed by taking the power of the signal (VENG²) and calculating the envelope of the VENG². By comparing this signal with FIG. 3, D, that shows the BP as recorded directly from the carotid artery (with a catheter inside the artery, connected to a pressure transducer), and FIG. 3, E, which shows the respiration pressure, it can be seen that the signal in B contains a signal “a”, which is strongly correlated with the BP pulse (BPP) occurring during each heartbeat, but also contain a slower component “b”, which is correlated with the respiration. To remove interference from the respiration (which can be more prominent than in the example), the VENG² envelope is further filtered to remove this signal, producing the signal C.

From the signal in C, the BPPs can be identified by an algorithm that identifies the peak in this BPP-related neural activity. When a new peak is identified it triggers the extraction of a segment of the neural signal, e.g. 300 ms to each side of the peak. To reduce noise in the recording, which is not related to the BP, the segments may be averaged for several BPPs, e.g. from the preceding 10 s, to produce a BPP-related profile of the neural signal.

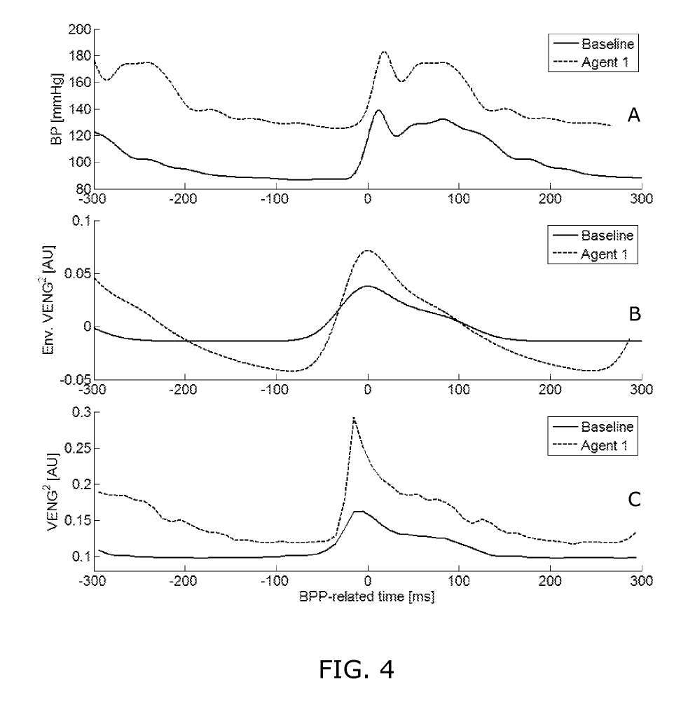

A shows BPP-related profiles of the BP, as measured from the carotid artery. FIG. 4, B shows BPP-related profiles of the VENG² envelope signal from C in FIG. 3.

FIG. 4, C shows alternative BPP-related profiles, which were extracted by using the method described above to extract segments of VENG². These segments were then averaged (and bin integrated). In each of the plots, the solid line represents an average of the “baseline” period, i.e. the time preceding the intervention. After this period, “Agent 1” (adrenaline) was infused in the animal, which resulted in a transient BP increase. In each of the plots, the dotted line represents a profile averaged for the 20 s when the BP was at its maximum. The increase in BP (FIG. 4, A) leads to a substantial increase in the amplitude of the VENG profiles (FIG. 4, B and C). From these profiles, information can be extracted, e.g. in the form of value of positive peak, negative peak, or amplitude (positive−negative peak).

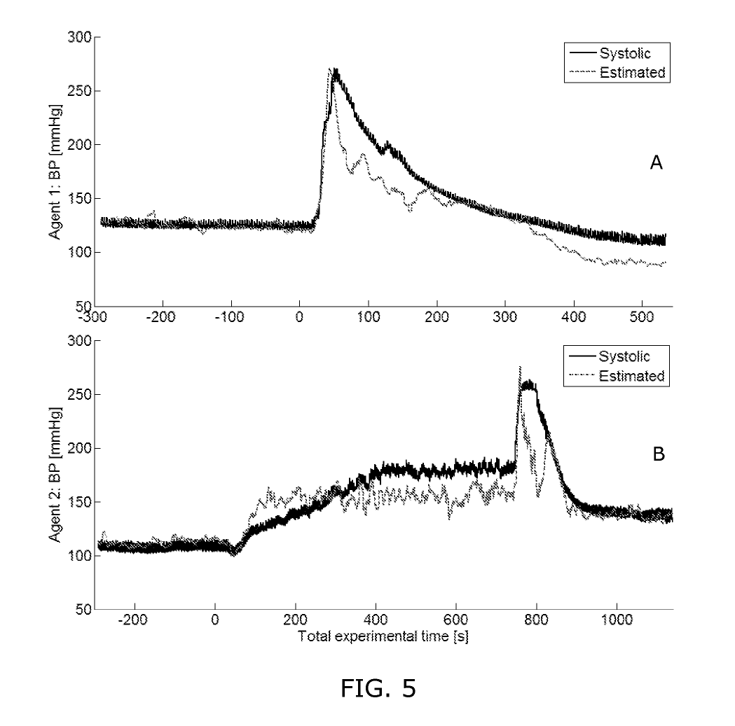

FIG. 5 shows an example of measured (systolic) and estimated BP during an experiment. The BPPs were identified as described above and used to trigger averaging of BPP-related neural segments, extracted from the VENG² envelope signal (FIG. 3, C and FIG. 4, B), for the preceding 10 s. From each average, the amplitude of the profile was extracted to define a feature for BP estimation. During the experiment the animal was infused with two different drugs, agent 1 (FIG. 5, A) and agent 2 (FIG. 5, B), that caused the BP to increase. In order to relate the feature extracted from the neural signal to real BP, it was calibrated using the data with agent 1 (FIG. 5, A).

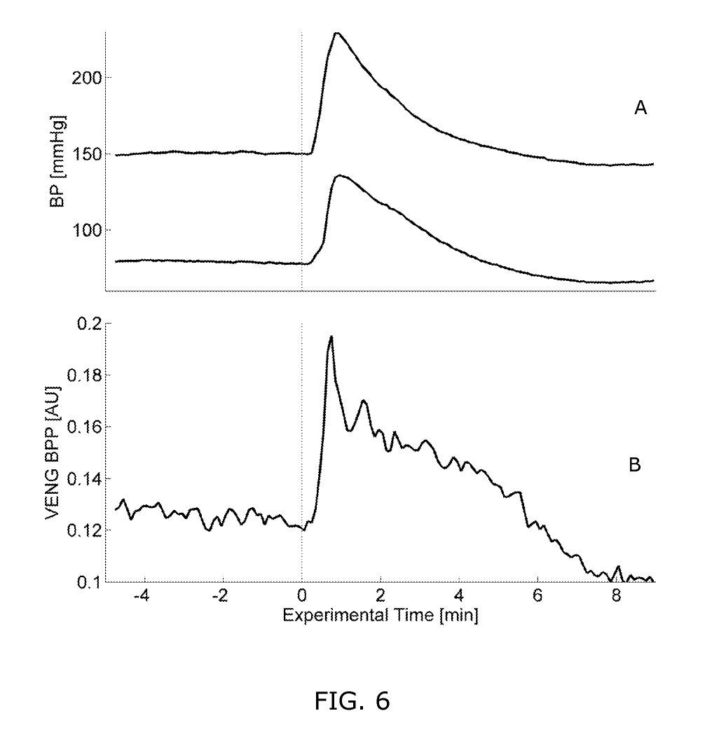

FIG. 6 shows the average of recordings made in seven pigs. BP was stable in the anaesthetized pigs for five minutes after which injection of agent 1 caused an increase in both systolic and diastolic BP (FIG. 6, A). The same neural feature that was used for BP estimation in the example of FIG. 5 was averaged across the seven animals (FIG. 6, B). The averaged development of the BP and the neural BP estimation feature in response to agent 1 were correlated. At the peak increase, BP was 58% higher than during the baseline period while the neural feature was 55% larger than the baseline average.

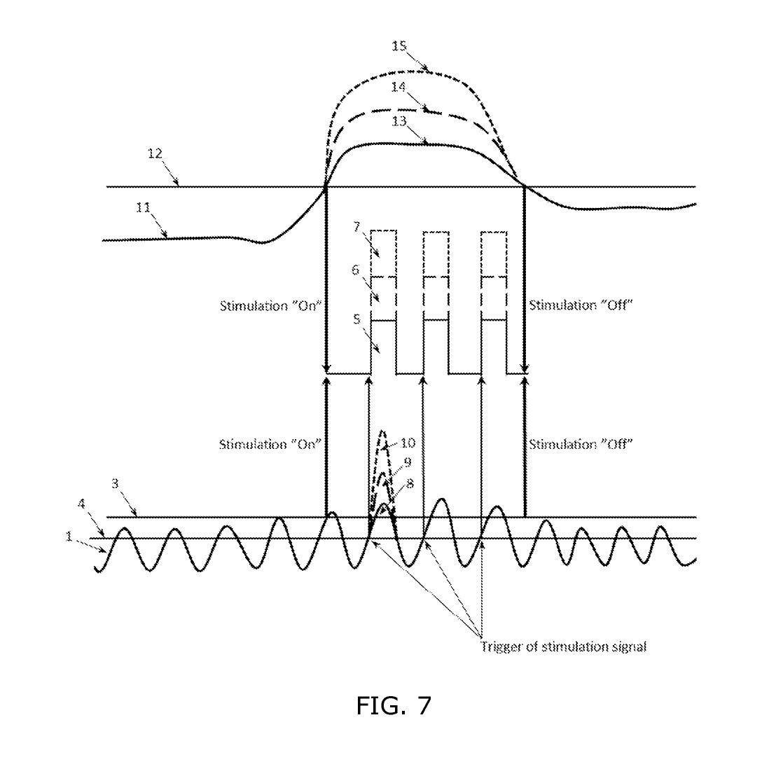

FIG. 7 shows how the BPP-related neural profiles (FIG. 7, 1) or the feature for BP estimation (FIG. 7, 11) can be used to control the stimulation unit of an implanted VNS or carotid body stimulation system for BP control. In a preferred embodiment, and as illustrated in FIG. 7, controlling of the stimulation unit comprises the following sequence of events:
i) activation of the <Stimulation “On”>/<Stimulation “Off”> functions,
ii) triggering of the actual stimulation signals, and
iii) adjustment of the strength of the stimulation signals relative to the peak-to-peak amplitude of the BPP-related neural profiles or to the value of the feature for BP estimation.

If BPP-related neural profiles (FIG. 7, 1) are used to control a VNS or carotid body stimulation unit, the <Stimulation “On”> function is activated when the peak-to-peak amplitude of the BPP-related neural profiles increases above a threshold indicative of BP higher than normal (FIG. 7, 3). Following the activation of the <Stimulation “On”> function, triggering of the actual stimulation signals occurs when a threshold indicative of a BPP-related neural profile (FIG. 7, 4) is reached. When the peak-to-peak amplitude of the BPP-related neural profiles decreases below the threshold indicative of BP higher than normal (FIG. 7, 3), the <Stimulation “Off”> function is activated.

In a preferred embodiment, the strength of the stimulation signals (situations 5, 6 and 7 in FIG. 7) is adjusted proportional to the peak-to-peak amplitude of the BPP-related neural profiles (situations 8, 9 and 10 in FIG. 7). The stimulation signals can be in the form of single electrical impulses, or of a train of electrical impulses. If the stimulation signals comprises single electrical impulses, the strength of the stimulation signals can be adjusted by changing the amplitude or the duration of the electrical impulses, or both the amplitude and the duration of the electrical impulses. If the stimulation signals comprises trains of electrical impulses, the strength of the stimulation signals can be adjusted by changing the amplitude, the duration and the frequency of the electrical signals, either alone or simultaneously for amplitude, duration and frequency, or in any combination of two of the mentioned stimulation parameters.

If the feature for BP estimation (FIG. 7, 11) is used to control a VNS or carotid body stimulation unit, the <Stimulation “On”> function is activated when the value of the feature for BP estimation increases above a threshold indicative of BP higher than normal (FIG. 7, 12). Following the activation of the <Stimulation “On”> function, triggering of the actual stimulation signals occurs when a threshold indicative of a BPP-related neural profile (FIG. 7, 4) is reached. In a preferred embodiment, the strength of the stimulation signals (situations 5, 6 and 7 in FIG. 7) is adjusted proportional to the value of the feature for BP estimation (situations 13, 14 and 15 in FIG. 7). The strength of the stimulation signals is adjusted as described above.

The nerve signal may be processed in various ways before the BPP-related profile is extracted as described above. The BPP-related profile may, e.g. be based on the rectified signal, signal power, variance, entropy, or frequency dependent features, e.g. extracted using Fourier transformation, autoregressive modeling, or wavelet analysis.

In addition to the embodiment described above, information from the ECG may be used to guide BPP identification, or extraction of the BPP-related profile may instead be based on triggers extracted from the ECG.

The method is not limited to the vagus nerve but can be used with neural signals from all nerves transmitting baroreceptor information.

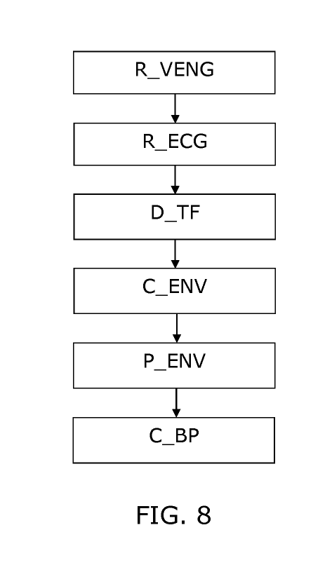

FIG. 8 shows steps of a method embodiment, e.g. for being performed as an executable program code in a processor of a device, e.g. an implantable device.

First step includes receiving a neural activity input recorded from a patient's Vagus nerve R_VENG, as receiving well as receiving of accordingly recorded ElectroCardioGraphy input determined on the patient R_ECG. The ECG is used for determining a trigger feature D_TF, and in response to this trigger feature, an envelope of the Vagus nerve activity input is calculated C_ENV. Next, this envelope is processed P_ENV, and based thereon a final step of calculating a measure indicative of the patient's blood pressure is performed C_BP. Such blood pressure output can be output to an external device, e.g. a mobile phone or the like, or it can be used for controlling a blood pressure control device in an open-loop manner, e.g. a for control of a Vagus nerve stimulation device.

In the following, special embodiments E1-E30 of the invention are defined.

E1. A device arranged to analyze an input signal (1) representing a neural activity of a nerve containing nerve fibers originating from baroreceptors from a patient, such as an input signal representing neural activity of the Vagus nerve of a patient, and to determine a value indicative of the patient's blood pressure (BP) based thereon, such as a value indicative of the patient's systolic blood pressure.
E2. Device according to E1, comprising an input unit (3) arranged to receive a neural activity signal (1) and to output a digital version thereof.
E3. Device according to E1 or E2, comprising a signal processing unit (4) arranged to analyze the neural activity data (1) and to extract a segment of the neural activity data (1) to emphasize variations occurring with the cardiac rhythm (2).
E4. Device according to E3, wherein the signal processing unit (4) is arranged to generate an averaged segment based on a plurality of segments of the neural activity data (1).
E5. Device according to any of E1-E4, comprising an estimation unit (6) arranged to estimate one or more blood pressure (BP) components from blood pressure pulse information (BPP) in the neural activity data (1).
E6. Device according to any of E1-E5, arranged to extract an envelope (VENG) of a neural activity signal (1), such as determining at least one of: a positive peak, a negative peak, and an amplitude, of the neural activity signal (1).
E7. Device according to any of E1-E6, comprising means for performing a calibration procedure, so as to increase precision of the value indicative of the patient's blood pressure (BP).
E8. Device according to any of E1-E7, the device comprises:
a. an input unit (3) for receiving and digitizing a neural signal (1), and possibly an Electro CardioGraphy signal (2),
b. a signal processing unit (4) for:
i. Preprocessing the neural signal (1) to emphasize Blood Pressure Pulse information, ii. Detecting from the processed neural signal a trigger feature marking each Blood Pressure Pulse.iii. Detecting at least one feature from the Electro CardioGraphy signal, such as R-peaks.
vi. In response to detecting a trigger feature, extracting a segment of processed neural signal around the trigger feature.
   c. a signal extraction unit (5) for:
i. Generating an averaged signal segment from a number of nerve signal segments.
ii. Extracting one or more Blood Pressure Pulse-related feature(s) from either the neural signal segment or the averaged neural signal segment.
   d. an estimation unit (6) for:
i. Calculating from the extracted Blood Pressure Pulse-related feature(s) an estimated value of one or more Blood Pressure components.
ii. Calibrating the system by extracting Blood Pressure-related feature(s) and estimating the value of one or more Blood Pressure components in normal Blood Pressure conditions to establish preset values for Blood Pressure components in normal conditions.
   iii. Comparing the estimated value of the Blood Pressure component(s) to preset intervals for use in a treatment modality.
   E9. Device according to any of E1-E8, wherein parts of or all of a preprocessing of the input signal (1) is performed by means of hardware components.
   E10. Device according to any of E1-E9, wherein information from the ECG is used to assist in identification of the BPP.
   E11. Device according to any of E1-E10, wherein BPP onset is used to trigger onset of a treatment modality.
   E12. Device according to any of E1-E11, wherein identification of the trigger feature is based on ECG instead of BPP.
   E13. Device according to any of E1-E12, wherein the nerve activity data is obtained from at least one nerve selected from: The vagus nerve (cranial nerve X), the glossopharyngeal nerve (cranial nerve IX), The carotid sinus nerve (Hering's nerve), and The aortic depressor nerve.
   E14. Device according to any of E1-E13, wherein nerve activity data is recorded from the whole nerve.
   E15. Device according to any of E1-E14, wherein nerve activity data is recorded from part of the nerve.
   E16. Device according to any of E1-E15, wherein the device further comprises or is connected to a treatment modality.
   E17. Device according to E16, wherein the treatment modality comprises a Vagus Nerve Stimulation system or a drug-delivery system.
   E18. Device according to any of E1-D17, comprising a nerve electrode arranged to sense said neural activity.
   E19. Device according to any of E1-E18, comprising a neural activity recorder arranged to record a sequence of a neural signal.
   E20. Device according to any of E1-E19, comprising a nerve stimulation unit arranged to generate an electric nerve stimulation signal.
   E21. Device according to E20, comprising a control unit arranged to control the nerve stimulation unit in response to the determined value indicative of the patient's blood pressure BP with the purpose of controlling the patient's blood pressure BP.
   E22. Device according to E21, wherein the nerve stimulation unit and the control unit are connected to form a closed-loop configuration.
   E23. A method for estimating a blood pressure of a patient, comprising
analyzing neural activity data, such as a neural signal, of a nerve containing nerve fibers originating from baroreceptors, such as the Vagus nerve, from a patient according to an analyzing algorithm, and
determining a value indicative of the patient's blood pressure (BP) based thereon.
   E24. Method according to E23, comprising determining an envelope of a nerve signal (VENG).
   E25. Method according to E23 or E24, comprising determining an envelope of a second powered version of a nerve signal (VENG²).
   E26. Method according to any of E23-E25, comprising deriving a trigger feature from ElectroCardioGraphy (ECG) data from the patient temporally aligned with the neural activity data.
   E27. Method according to E26, comprising processing the neural activity data by emphasizing blood pressure pulse information by means of said trigger feature.
   E28. Method according to any of E23-E27, comprising determining a measure of a systolic blood pressure of the patient based on said neural activity.
   E29. Method according to any of claims E23-E28, comprising filtering the neural activity data, such as low pass filtering a sequence of a neural signal prior to determining the value indicative of the patient's blood pressure (BP).
   E30. Computer executable program code arranged to perform the method according to any one of 22-28.
   E31. Method of treating hypertension using the device according to any of E1-E22 or the method according to any of E23-E29.

To sum up, the invention provides a device and a method for analyzing an input signal 1 representing a neural activity of a nerve containing nerve fibers originating from baroreceptors from a patient. E.g. neural activity recorded with an electrode implanted to detect Vagus nerve activity. The invention is based on the insight that this neural activity can be used to determine a value indicative of 35 the patient's blood pressure BP. An ECG signal 2 can be used for deriving a trigger feature for processing of the nerve activity data, e.g. to arrive at a profile of the patient's Blood Pressure Pulse. E.g. the device can include a Vagus nerve stimulation unit which is controlled in response to an output from the calculation of blood pressure BP, and/or features related to the Blood Pressure Pulse.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1.-48. (canceled)

49. A device arranged to analyze an input signal (1) representing a neural activity of a nerve containing nerve fibers originating from baroreceptors from a patient, and to determine a value indicative of the patient's blood pressure (BP) based thereon.

50. Device according to claim 49, comprising a signal processing unit (4) arranged to analyze the digital version of the input signal and to extract a segment of the neural activity data (1) represented therein, to emphasize variations occurring with the cardiac rhythm (2).

51. Device according to claim 49, comprising an estimation unit (6) arranged to estimate one or more blood pressure (BP) components from Blood Pressure Pulse information in the neural activity data represented in the input signal (1).

52. Device according to claim 49, arranged to extract an envelope (VENG) of a neural activity data represented in the input signal (1).

53. Device according to claim 52, wherein said envelope is extracted by determining at least one of: a positive peak, a negative peak, and an amplitude, of the neural activity data represented in the input signal (1).

54. Device according to claim 52, wherein said Blood Pressure Pulse information is calculated in response to said envelope of the neural activity signal Blood Pressure.

55. Device according to claim 49, comprising means for performing a calibration procedure, so as to increase precision of the value indicative of the patient's blood pressure (BP).

56. Device according to claim 49, wherein the device comprises:
a signal processing unit arranged for preprocessing the neural signal (1) to emphasize Blood Pressure Pulse information,
a signal extraction unit arranged extracting one or more Blood Pressure Pulse-related feature(s) from either a neural signal segment or an averaged neural signal segment, and
an estimation unit arranged for calculating from the at least one of the extracted one or more Blood Pressure Pulse-related feature(s), an estimated value of one or more Blood Pressure components (BP).

57. Device according to claim 56, wherein the signal extraction unit is arranged to perform a segmentation of the input signal (1), and to generate an averaged neural signal segment in response to an output of said segmentation.

58. Device according to claim 49, wherein Blood Pressure Pulse onset is used to trigger onset of a treatment modality.

59. Device according to claim 49, wherein the device further comprises or is connected to a treatment modality, and wherein the treatment modality comprises a Vagus Nerve Stimulation system or a drug-delivery system.

60. Device according to claim 49, comprising a nerve stimulation unit arranged to generate an electric nerve stimulation signal.

61. Device according to claim 60, comprising a control unit arranged to control the nerve stimulation unit in response to one or more Blood Pressure Pulse-related features.

62. Device according to claim 61, wherein the control unit is arranged to control the nerve stimulation unit so as to adjust a strength of the electric nerve stimulation signal relative to an amplitude of a Blood Pressure Pulse-related feature.

63. Device according to claim 62, wherein the Blood Pressure Pulse-related feature comprises a Blood Pressure Pulse-related profile, wherein the Blood Pressure Pulse-related profile is calculated based on at least one of: a rectified signal, a calculated signal power, a calculated variance, a calculated entropy, or a frequency dependent feature.

64. Device according to claim 61, wherein the nerve stimulation unit and the control unit are connected to form a closed-loop configuration.

65. A method for estimating a blood pressure of a patient, comprising
analyzing neural activity data of a nerve containing nerve fibers originating from baroreceptors, from a patient, according to an analyzing algorithm, and
determining a value indicative of the patient's blood pressure (BP) based thereon.

66. Method according to claim 65, comprising deriving a trigger feature from ElectroCardioGraphy (ECG) data from the patient temporally aligned with the neural activity data, and comprising processing the neural activity data by emphasizing Blood Pressure Pulse information by means of said trigger feature.

67. Method according to claim 65, comprising filtering the neural activity data, such as low pass filtering a sequence of a neural signal prior to determining the value indicative of the patient's blood pressure (BP).
